(19) United States
(12) Patent Application Publication
(10) Pub. No.: US 2007/0135833 A1
(43) Pub. Date: Jun. 14, 2007

(54) RADIOPAQUE EMBOLIC PROTECTION FILTER MEMBRANE

(75) Inventors: Dnyanesh Talpade, Plymouth, MN (US); Peter Hirshman, Minneapolis, MN (US); Yem Chin, Burlington, MA (US); Sheng-Ping Zhong, Shrewsbury, MA (US); Kinh-Luan D. Dao, Randolph, MA (US); James G. Hansen, Coon Rapids, MN (US); Brian J. Lowe, Zimmerman, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/672,323

(22) Filed: Feb. 7, 2007

Related U.S. Application Data
(63) Continuation of application No. 10/400,751, filed on Mar. 27, 2003.

(51) Int. Cl. A61M 29/00

(52) U.S. Cl. 606/200

(57) ABSTRACT

An embolic protection filter assembly and method of making the same. In at least some embodiments, the present invention relates to embolic protection filters having at least one radiopaque component.

This application is a continuation of U.S. application Ser. No. 10/400,751 filed Mar. 27, 2003.

### FIELD OF THE INVENTION

The present invention pertains to embolic protection filter devices. More particularly, the present invention pertains to embolic protection filters having a radiopaque marker.

### BACKGROUND

Heart and vascular disease are majors problem in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

### BRIEF SUMMARY

The present invention pertains to embolic protection. In some embodiments, an embolic protection filter assembly includes an elongate shaft and a filter coupled to the shaft. The filter may include at least one radiopaque component that can enhance the ability of a clinician to visualize the filter. The radiopaque component may be mixed or distributed throughout a polymer and the filter can be formed, for example, by dipping a mandrel into the mixture. Alternatively, the filter may be formed from a plurality of layers and one or more of the layers may include a radiopaque material.

### BRIEF DESCRIPTION OF THE DRAWINGS

FIG. 1 is a prospective view of an example embolic protection filter assembly;

FIG. 2 is a prospective view of a forming member appropriate for forming an embolic protection filter;

FIG. 3 is a plan view of a radiopaque filter material coupled to an embolic protection filter frame and a mandrel; and

FIG. 4 is a plan view of another example radiopaque filter material disposed adjacent a mandrel.

### DETAILED DESCRIPTION

The following description should be read with reference to the drawings wherein like reference numerals indicate like elements throughout the several views. The detailed description and drawings illustrate example embodiments of the claimed invention.

When a physician performs an intravascular intervention such as angioplasty, atherectomy, and the like, embolic debris may dislodge from the blood vessel. This embolic debris can travel in the bloodstream and impair blood flow, possibly leading to tissue damage. In order to account for this embolic debris, a number of filtering devices have been developed that can be used in conjunction with a debris-producing intervention. These embolic protection filters can be disposed in the blood vessel downstream of the treatment site and expanded to capture the debris.

Often it is desirable for the clinician to be able to track and/or visualize the location of the filter within the body. One way to accomplish this is to couple a radiopaque marker to the guidewire adjacent the filter or to the mouth of the filter frame. Although the former strategies are effective, they tend to allow only a portion of the filter or a region adjacent the filter to be visualized. It may be desirable for the filter to be visualized more completely so that the clinician can perform an intravascular intervention with heightened precision. In at least some embodiments, the present invention relates to embolic protection filters (and methods of making the same) that allow for improved filter visualization.

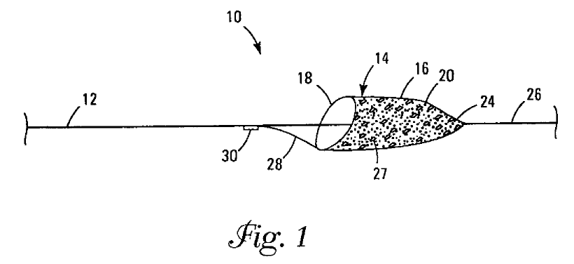

FIG. 1 illustrates an embolic protection filter assembly 10 including an elongate shaft or guidewire 12 having an embolic protection filter 14 coupled thereto. Filter 14 includes a filter material 16 coupled to a filter frame 18 (best seen in FIG. 3). Filter material 16 includes one or more radiopaque components 20 shown as being homogeneously distributed throughout filter material 16. Having radiopaque components 20 distributed throughout filter material 16 allow essentially all of filter 12 to be visualized by the clinician.

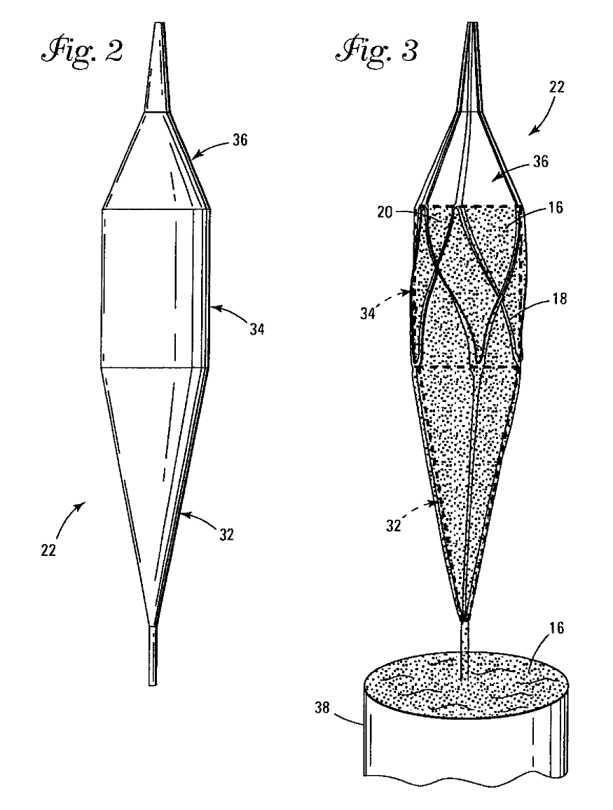

Radiopaque components 20 may include one or more radiopaque materials that are disposed, distributed, doped, or otherwise a component of filter material 16. Radiopaque materials are understood to be materials capable of producing a relatively bright image on a fluoroscopy screen or another imaging technique during a medical procedure. This relatively bright image aids the user of assembly 10 in determining its location. Radiopaque materials can include, but are not limited to, bismuth subcarbonate, iodine, gold, platinum, palladium, tantalum, tungsten or tungsten alloy, and the like. In the embodiment show in FIG. 1, radiopaque components 20 are illustrated as being distributed throughout a generally polymeric filter material 16. The material can also be MRI compatible. This manner of distribution can be achieved, for example, by mixing radiopaque components 20 with a molten or otherwise liquefied filter material 16 and then dipping a filter mandrel (an appropriate example is mandrel 22 illustrated in FIG. 2) into the mixture. Other techniques such as blow or vacuum molding, stretch forming, or spraying can be used.

The result of dip-molding mandrel 22 into a mixture of radiopaque components 20 and filter material 16 may result in filter 14 that has radiopaque components 20 distributed essentially homogeneously throughout. Although the distribution has been described as being homogeneous and throughout, it can be appreciated that the precise distribution may be altered in different embodiments. For example, it may be desirable for a greater portion of radiopaque components 20 to be disposed at a particular part of filter 14 such as a narrowed distal end 24 thereof. Because the amount of filter material 16 disposed at distal end 24 decreases (as the size of filter 12 decreases), having a greater concentration of radiopaque components 20 adjacent narrowed distal end 24 may enhance visualization of distal end 24.

As suggested above, filter material 16 may be generally comprised of a polymer or combination of polymers. Some examples of suitable polymers include polyurethane, polyester-ether (for example a polyester-ether elastomer such as ARNITEL™ available from DSM Engineering Plastics), polyester (for example a polyester elastomer such as HYTREL® available from DuPont), or linear low density polyethylene (for example REXELL®), polypropylene (PP), polyvinylchloride (PVC), polytetrafluoroethylene (PTFE), polyether-ether ketone (PEEK), polyimide, polyamide (for example, DURETHAN® available from Bayer or CRISTAMID® available from Elf Atochem), elastomeric polyamides, polyphenylene sulfide (PPS), polyphenylene oxide (PPO), polysufone, perfluoro(propyl vinyl ether) (PFA), block polyamide-ethers, polyether block amide (PEBA, for example available under the trade name PEBAX®), polycarbonate urethane (for example, CORETHANE® available from Corvita Corp.), silicones, nylons, polyethylene, Marlex high-density polyethylene, and the like, or mixtures, combinations, or copolymers thereof. The polymer may be doped or include radiopaque component 20 or be combined and/or mixed with radiopaque component 20 as described above. As a result, filter material 16 has the desired level of radiopaque components 20 that allow filter 14 has the desired radiopacity.

Shaft 12 may comprise a guidewire, catheter, tube, or the like and can be made of any suitable material including metals, metal alloys, polymers, or the like, or combinations or mixtures thereof. Some examples of suitable metals and metal alloys include stainless steel, such as 304v stainless steel; nickel-titanium alloy, such as nitinol, nickel-chromium alloy, nickel-chromium-iron alloy, cobalt alloy, or the like; or other suitable material. The entire shaft 12 can be made of the same material, or in some embodiments, can include portions or sections made of different materials. In some embodiments, the material used to construct shaft 12 is chosen to impart varying flexibility and stiffness characteristics to different portions of shaft 12. For example, the material used to construct a proximal region can be relatively stiff for pushability and torqueability (e.g., straightened 304 stainless steel wire), and the material used to construct a distal region can be relatively flexible by comparison for better lateral trackability and steerability (e.g., a straightened super elastic or linear elastic alloy such as nickel-titanium wire).

Filter 14 may be coupled to shaft 12 near a distal end 26 thereof, however, it can be appreciated that filter 14 could be disposed at essentially any position along shaft 12. For example, shaft 12 can pass through a portion of filter 14 so that the distal end of filter 14 and frame 18 (and/or struts 28 extending from frame 18) can be attached to shaft 12 as shown in FIG. 1. However, it can be appreciated that a number of different styles or configurations of filter 14 can be utilized without departing from the spirit of the invention. In general, filter 14 operates between a first generally collapsed configuration and a second generally expanded configuration for collecting debris in a body lumen. Frame 18 may be comprised of a “self-expanding” shape-memory material such as nickel-titanium alloy (to bias filter 14 to be in the second expanded configuration). Additionally, frame 18 may include a radiopaque material or include, for example, a radiopaque wire disposed about a portion thereof. Filter material 16 can be drilled (for example, formed by known laser techniques) or otherwise manufactured to include at least one opening 27. The holes or openings 27 are sized to allow blood flow therethrough but restrict flow of debris or emboli floating in the body lumen or cavity. One or more struts 28 may extend between frame 18 and shaft 12 and be coupled to shaft 12 by a coupling 30. Coupling 30 may be one or more windings of struts 28 about shaft 12 or be a fitting disposed over an end of struts 28 to attach it to shaft 12.

FIG. 2 is a prospective view of a forming member such as a mandrel 22, which is an appropriate example of a mandrel for forming filter 14. Mandrel 22 includes a tapered or necked distal region 32, a mid-region 34, and a proximal region 36 that may also be tapered. A central channel or lumen (not shown) may be formed through the center along the longitudinal axis of mandrel 22 that can be used, for example, as a location for shaft 12 to be disposed during the manufacturing of filter 14. Mandrel 22 may be comprised of any appropriate material including metals and polymers.

Frame 18 may be disposed over mandrel 22 as shown in FIG. 3. In some embodiments, frame 18 is disposed over distal region 32 and mid-region 34. The portion of frame 18 disposed adjacent distal region 32 may be attached to shaft 12. Additionally, struts 28 may extend from frame 18 over proximal region 36 in a manner amenable to having struts 28 being attached to shaft 12.

In some embodiments, mandrel 22, together with or independently of frame 18, can be dipped into a container 38 of filter material 16. Frame 18 may be coated or pre-treated with a tie-layer or adhesive such as thixon. As described above, filter material 16 may be molten and include radiopaque component 20. When mandrel 22 is dipped into and then removed from container 38, a layer of filter material 16 remains disposed on and generally conforms to the shape of mandrel 22. Filter material 16 can be allowed to solidify. The now solidified layer of filter material 16 and frame 18, ultimately, will define filter 14.

The thickness of filter material 16 disposed on mandrel 22 can be altered in different embodiments. For example, it is believed that holding mandrel 22 within container 38 for an extended period of time may allow a greater amount of filter material 16 to become disposed adjacent mandrel 22. Alternatively, changing the speed of dipping (either the speed of entry or withdrawal from container 38), the polymer used for filter material 16, the temperature of filter material 16, or other conditions may also play a role in determining the thickness. In some embodiments, the dipping step may be repeated additional times to increase thickness or incorporate other desired properties. For example, an initial dipping step may include dipping mandrel 22 into a non-radiopaque filter material, followed by dipping into filter material 16 (including radiopaque component 20). A final dipping step may also be performed to effectively “sandwich” or embed the radiopaque layer.

Additional manufacturing steps may also be performed to complete the manufacturing of filter 14. For example, filter material 16 and frame 18 may be attached removed from mandrel 22 and attached to shaft 12. Additionally, a number of holes 27 may be drilled in filter material 16. When complete, assembly 10 may be used to facilitate an intravascular intervention. For example, assembly 10 may be loaded within a delivery sheath and advanced through the vasculature to a location adjacent (i.e., “downstream”) of a lesion. The sheath can be retracted, allowing frame 18 to expand filter 12, and a therapeutic or diagnostic medical device, for example an angioplasty or atherectomy catheter, can be advanced over shaft 12. Debris generated by the medical device can be capture by filter 14 and later removed from the body.

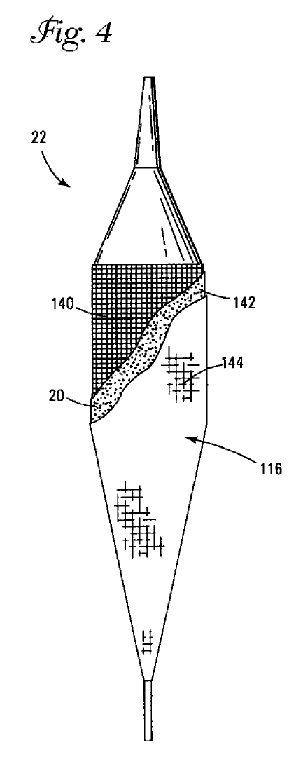

FIG. 4 is a plan view of another example radiopaque filter material 116 coupled disposed adjacent mandrel 22. Filter material 116 is appropriate for forming filter 14 and is similar to filter material 16 except that it includes a plurality of layers that can be wrapped, spun, or braided about mandrel 22. One or more of the individual layers may include radiopaque component 20. For example, filter material 116 may include a first layer 140, a second layer 142, and a third layer 144, and second layer 142 may comprise a radiopaque wire.

The multiple layers (e.g., layers 140, 142, and 144) each may include a polymer, for example polycarbonate urethane. In some embodiments, one or more of the layers 140/142/144 may include radiopaque component 20 in the form described above, in the form of a radiopaque wire, or include radiopaque wire that is embedded within a polymer. The embedded radiopaque component 20 and polymer (or other suitable structure) may then be spun or otherwise disposed about mandrel 22. Alternatively, one of the layers, for example second layer 142, may include radiopaque component 20 and may be disposed between two layers, for example first layer 140 and third layer 144.

The arrangement or configuration of layers 140/142/144 may be altered to incorporate a number of desired properties. For example, layers 140/142/144 can be braided or intertwined either with each other or with themselves, which may enhance the strength of filter 14. In some embodiments, including layers 140/142/142 may obviate the need for frame 18. Alternatively, the thickness of each individual layer may vary. For example, layer 142 may be thicker than layer 140. In some embodiments, layers 140/142/144 can be configured so as to define holes 27. For example, layers 140/142/144 may be braided and holes 27 may be defined within the braids. This strategy may allow for greater control of the diameter and/or distribution of holes 27.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method of manufacturing an embolic protection filter assembly, comprising the steps of:
providing a forming member;
disposing a filter frame over at least a portion of the forming member;
providing a filter material, the filter material comprising a polymeric material including one or more radiopaque components mixed throughout the polymeric material;
disposing the filter material over at least a portion of the filter frame;
wherein the one or more radiopaque components are non-homogenously mixed throughout the polymeric material.

2. The method of claim 1, wherein the step of providing a filter material includes providing a molten polymer doped with a radiopaque substance.

3. The method of claim 2, wherein the forming member includes a mandrel.

4. The method of claim 1, wherein the step of disposing the filter material over at least a portion of the filter frame includes disposing a first layer of the filter material over at least a portion of the filter frame and disposing a second layer of a second filter material adjacent the first layer.

5. The method of claim 4, further comprising the step of disposing a third layer of a third filter material adjacent the first layer and the second layer.

6. The method of claim 4, wherein at least one of the first layer, second layer, or third layer includes a radiopaque material.

7. A method of manufacturing an embolic protection filter, comprising the steps of:
providing a mandrel member including a filter-shaped region;
providing a liquefied filter material doped with a radiopaque material;
applying the filter material to the filter-shaped region of the mandrel member, wherein a filter portion of the filter material generally conforms to the shape of the filter-shaped region; and
allowing the filter portion to solidify;
wherein the one or more radiopaque components are non-homogenously distributed throughout the filter material.

8. The method of claim 7, further comprising the step of separating the filter portion from the filter-shaped region.

9. The method of claim 7, further comprising the step of forming a plurality of holes in the filter portion.

10. The method of claim 7, further comprising the step of coupling the filter portion to an elongate shaft.

11. An embolic protection filter assembly, comprising:
a filter frame having a body portion and a mouth portion;
a filter material coupled to the filter frame;
wherein the filter material includes a polymeric material having at least one radiopaque component mixed with the polymeric material adjacent the body portion; and
wherein said at least one radiopaque component is non-homogenously mixed with the polymeric material.

12. The embolic protection filter assembly of claim 11, wherein the filter frame comprises nickel-titanium alloy.

13. The embolic protection filter assembly of claim 11, wherein the filter material includes a plurality of layers and wherein the radiopaque component comprises at least one of the layers.

14. The embolic protection filter assembly of claim 11, wherein the radiopaque component includes bismuth.

15. The embolic protection filter assembly of claim 11, wherein the radiopaque component includes iodine.

16. The embolic protection filter assembly of claim 11, wherein the filter material includes polyurethane.

17. A method of manufacturing an embolic protection filter assembly, comprising the steps of:
providing a forming member;
positioning a filter frame over the forming member;
disposing a first layer of filter material over at least a portion of the forming member;
disposing a second layer of filter material over at least a portion of the first layer, the second layer of filter material including a radiopaque component; and
disposing a third layer of filter material over at least a portion of the second layer, the third layer of filter material not including a radiopaque components
wherein the first layer, second layer and third layer form a filter portion.

18. The method of claim 17, wherein the step of disposing a first layer of filter material over at least a portion of the forming member includes braiding.

19. The method of claim 17, further comprising the step of coupling the filter portion to an elongate shaft.

20. An multi-layer embolic protection filter, comprising:
a filter frame; and
a filter portion coupled to the filter frame, the filter portion including:
a first polymeric layer;
a second layer disposed adjacent the first layer the second layer including a radiopaque component; and
a third layer disposed adjacent the first layer and the second layer, the third layer not including a radiopaque component.

21. The multi-layer embolic protection filter of claim 20, wherein the radiopaque component includes a radiopaque wire encapsulated in a polymer.

22. The multi-layer embolic protection filter of claim 20, wherein the radiopaque component includes bismuth.

23. The multi-layer embolic protection filter of claim 21, wherein the radiopaque component includes platinum.

24. An embolic protection filter assembly, comprising:
a filter frame; and
a filter material coupled to the filter frame, the filter material including a proximal section, a distal section, and the filter material comprising a polymeric material including a number of radiopaque components mixed throughout the polymeric material throughout the proximal and distal sections;
wherein the concentration of radiopaque components is greater at the distal section of said filter material than at the proximal section thereof.

25. An embolic protection filter assembly, comprising:
a filter frame; and
a filter portion coupled to the filter frame, the filter material having at least one layer of filter material comprising a polymeric material including one or more radiopaque components mixed throughout the polymeric material;
wherein said at least one layer of filter material is interposed between two or more additional layers of filter material dissimilar from said at least one layer of filter material.
